(19) United States
(12) Patent Application Publication
(10) Pub. No.: US 2025/0319310 A1
(43) Pub. Date: Oct. 16, 2025

(54) Pain Mitigation Device

(71) Applicants: Bryant George, New Orleans, LA (US); Herman J. Novak, Maryville, TN (US)

(72) Inventors: Bryant George, New Orleans, LA (US); Herman J. Novak, Maryville, TN (US)

(73) Assignee: Transitions LA, LLC, Metairie, LA (US)

(21) Appl. No.: 19/177,124

(22) Filed: Apr. 11, 2025

Related U.S. Application Data
(60) Provisional application No. 63/633,511, filed on Apr. 12, 2024.

(51) Int. Cl. A61N 1/36; A61N 1/05; G16H 20/30

(52) U.S. Cl. CPC A61N 1/36021; A61N 1/0502; A61N 1/36031; G16H 20/30

(57) ABSTRACT

A nerve stimulation device using AI for percutaneous vagal nerve and auricular nerve neuromodulation. A processor receives input parameters pertaining to physiological conditions of a patient, and processes the input parameters using AI to specify output parameters of a signal, a waveform generator to receive the output parameters and create the signal having the output parameters, an amplifier to receive the signal and amplify it to a desired level, an output to receive the signal from the amplifier, a lead to receive the signal from the output, and a probe to receive the signal from the lead and deliver the signal to a nerve of the patient.

### PRIORITY

This application claims priority on U.S. provisional patent application Ser. No. 63/633,511 filed 2024 Apr. 12, the entirety of the disclosure of which is incorporated herein by reference as if laid out in its entirety.

### FIELD

This invention relates to the field of pain mitigation. More particularly, this invention relates to a device that mitigates pain using an electrical stimulation.

### INTRODUCTION

Pain, regardless of its underlying causes, is a serious medical condition, and tends to compound the underlying issues from which it arises. For example, the understandable fear of pain, common to most people, can actually be a deterrent to address the underlying condition because of the fear of increasing the pain. Further, pain tends to result in a patient changing their physical behavior to some degree, which compensation can place undue stress on other parts of the body, resulting in damage to and additional pain from those compensating members.

Traditional methods of alleviating pain include surgery, therapy, and medication. Each has its associated problems. For example, for surgery to be effective in the reduction of pain, there must be a known method to alleviate the underlying condition. However, for a condition such as headache, there is no generally acknowledged surgical procedure for elimination of the condition. Therefore, surgery is an ineffective method to treat headache pain, at this point in time.

Further, while therapy tends to provide some amount of relief for the pain associated with muscular, joint, and connective tissue issues, it is not always effective for such, and is typically of no benefit for other types of pain-producing issues. Finally, medication can be effective to treat almost any kind and any level of pain, but it often comes at either the risk of addiction to the medication, which can be painful of itself to overcome, or give rise to an increasing tolerance to the medication, which makes it less effective against the pain over time.

What is needed, therefore, is a system for the mitigation of pain that tends to reduce issues such as those introduced above, at least in part.

### SUMMARY

The above and other needs are met by a nerve stimulation device with a processor to receive input parameters pertaining to physiological conditions of a patient, and processes the input parameters using AI to specify output parameters of a signal, a waveform generator to receive the output parameters and create the signal having the output parameters, an amplifier to receive the signal and amplify it to a desired level, an output to receive the signal from the amplifier, a lead to receive the signal from the output, and a probe to receive the signal from the lead and deliver the signal to a nerve of the patient.

In various embodiments according to this aspect of the disclosure, the physiological conditions include at least one of temperature, heart rate, HRV, blood pressure, electrical impulses of the patient's nervous system, blood oxygenation, hydration, and brain activity. Some embodiments include sensors for measuring the physiological conditions. Some embodiments include a radio for receiving at least one of the physiological conditions, power for the device, and operating instructions. In some embodiments, the lead comprises a signal lead and a ground lead. In some embodiments, the lead comprises multiple leads and the signal is applied to each of the multiple leads. In some embodiments, the lead comprises multiple leads and a different signal is applied to each of the multiple leads.

In some embodiments, the output parameters include at least one of location of signal delivery, signal intensity, signal waveform shape, signal frequency, signal cadence, signal duration, and signal amplitude. In some embodiments, the probe is a tri-tip probe that pierces the patient's epidermis and delivers the signal at an interface between the patient's epidermis and dermis. In some embodiments, the probe delivers the signal to at least one of the patient's auriculotemporal nerve, trigeminal nerve, and vagal nerve.

According to another aspect of the disclosure there is described a method of reducing pain in a patient, by receiving input parameters pertaining to physiological conditions of the patient, and processing the input parameters using AI to specify output parameters of a signal, receiving the output parameters with a waveform generator and creating the signal having the output parameters, receiving the signal with an amplifier and amplifying the signal to a desired level, receiving the signal from the amplifier with an output, receiving the signal from the output with a lead, receiving the signal from the lead with a probe, and delivering the signal from the probe to a nerve of the patient, the signal providing neuromodulation to the nerve and reducing the pain in the patient.

In various embodiments according to this aspect of the disclosure, the physiological conditions include at least one of temperature, heart rate, HRV, blood pressure, electrical impulses of the patient's nervous system, blood oxygenation, hydration, and brain activity. Some embodiments include measuring the physiological conditions with sensors. Some embodiments include receiving at least one of the physiological conditions, power for the device, and operating instructions with a radio. In some embodiments, the signal is applied to each of multiple leads. In some embodiments, a different signal is applied to each of multiple leads.

In some embodiments, the output parameters include at least one of location of signal delivery, signal intensity, signal waveform shape, signal frequency, signal cadence, signal duration, and signal amplitude. In some embodiments, the signal is delivered at an interface between the patient's epidermis and dermis. In some embodiments, the signal is delivered to at least one of the patient's auriculotemporal nerve, trigeminal nerve, and vagal nerve. In some embodiments, the pain includes pain from at least one of post operative shoulder surgery, post operative knee surgery, post operative cardiovascular surgery, post operative caesarian section surgery, drug detox, migraine headaches, pediatric irritable bowel, and diabetic neuropathy.

### BRIEF DESCRIPTION OF THE DRAWINGS

Further advantages of the invention are apparent by reference to the detailed description when considered in conjunction with the figures, which are not to scale so as to more clearly show the details, wherein like reference numbers indicate like elements throughout the several views, and wherein:

FIG. 1 is a functional block diagram for a device according to an embodiment of the present disclosure.

FIG. 2 is a first embodiment for a device according to the present disclosure.

FIG. 3 is a second embodiment for a device according to the present disclosure.

### DESCRIPTION

### General Overview

Embodiments of the device described herein generally relate to a pain mitigation device, which operates as a percutaneous nerve field stimulator that delivers electrical stimulation to one or more nerve, such as auricular or vagal nerves, with at least one of a user-selectable and AI-selectable neuromodulation, including voltage, timing, amplitude, frequency, duration, and waveform.

The device generally resides on or behind the ear and, in various embodiments, has detachable probes that connect to the patient's skin, where the probes include, in various embodiments, at least one voltage delivery probe and at least one ground probe. The ground probe makes surface contact with the skin, and the voltage delivery probes deliver the signal, in some embodiments, at the interface between the epidermis and the dermis, generally in the vicinity of the papillary and reticular layers.

In one embodiment, the device reads the patient's autonomic nervous system via heart rate variability (HRV), which is sensed either by the device itself, or which the device receives as an input from an external sensor. The HRV data (and any other data used by the device, as explained in greater detail hereinafter) is analyzed by either AI or a health care provider, who then sets the electrical stimulation parameters.

Various other embodiments deal with power sources, types of probes, color of leads, lead bundling, and programming interfaces.

### Electronic Control System

With reference now to the drawings, there are depicted all of the claimed elements of the various embodiments, although all claimed embodiments might not be depicted in a single drawing. Thus, it is appreciated that not all embodiments include all of the elements as depicted, and that some embodiments include different combinations of the depicted elements. It is further appreciated that the various elements can all have many different configurations, and are not limited to just the configuration of a given element as depicted. As indicated above, the elements of the drawings as depicted are not to scale, even with respect one to another, and relative size or thickness of one element cannot be determined by the aspect ratios of that element or with reference to any dimension of another element.

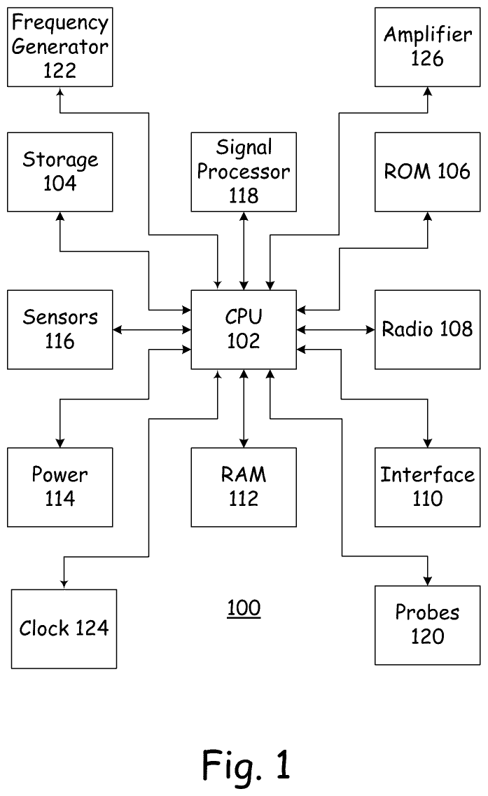

With reference now to FIG. 1, there is depicted one embodiment of a computerized device 100 capable of performing the functions as generally described herein. The apparatus 100 is at least one of a special purpose computing device, a tablet computer, a smart phone, a smart watch, a component level processor, an application specific integrated circuit, or some other computing device.

As used herein, the word module refers to a combination of both software and hardware that performs at least one designated function. Thus, in different embodiments, various modules might share elements of the hardware as described herein, and in some embodiments might also share portions of the software that interact with the hardware. In some other embodiments, a given module might be spread across different computer platforms. The various elements of device 100 as depicted in FIG. 1 are all modules of the device 100.

In some embodiments, the sensors 116 include devices for measuring at least one of temperature, heart rate, heart rate variability (HRV), blood pressure, electrical impulses of the nervous system, blood oxygenation, hydration, and brain activity. In some embodiments one or more of these sensors 116 are at least one of built-in to the device 100, connected directly to the device 100, and communicate with the device 100 wirelessly.

The radio module 108 provides a gateway for the communication of data and instructions between the device 100 and other sensors, probes, computing devices, networks, or data storage modules. In some embodiments, the radio 108 enables data communication over a wireless connection, including at least one of RFID, Bluetooth, UWB, cellular, Wi-Fi, Zigbee, and Z-Wave. In some embodiments the radio 108 receives programming instructions for the device 100.

In some embodiments, the device 100 is locally under the control of the central processing unit 102, which controls and utilizes the other modules of the device 100 as described herein. Also included in some embodiments is a frequency generator 122 and a clock or timing circuit 124. In various embodiments the signal produced by the device 100 and delivered to the patient can be at least one of a square wave, a sinusoidal wave, a stepped wave, and a sawtooth wave. These can be produced and adjusted, in various embodiments, by at least one of the CPU 102, frequency generator 122, and timing circuit 124. Some embodiments include an amplifier 126, to amplify the signals delivered to the probes 120 as desired.

The device 100 as depicted in FIG. 1 includes, in some embodiments, a non-transitory, computer-readable, data storage medium module 104 such as a flash drive, or some other relatively long-term data storage device. A read-only memory module 106 contains, for example, basic operating instructions for the operation of the device 100. An interface module 110 includes, for example, keyboards, speakers, microphones, cameras, displays, and touchpads, and provides means by which the user can interact with the device 100. In some embodiments, the interface module 110 includes a low voltage indicator, such as an LED 308, that indicates that a low charge is all that remains on the power module 114. In some embodiments, when the voltage of the power module 114 is below a given set point, the low voltage indicator 308 changes state, such as at least one of illuminates, changes color, changes intensity, and changes illumination patterns. These interface modules 110 connect directly to the device 100 in some embodiments, and in other embodiments connect to the device 100 using other means, such as the radio 108.

The probes 120 make direct electrical contact with the patient, as generally described herein, and include at least one ground probe 206 and at least one signal probe. In various embodiments, the probe tips 120 include at least one of barbs, spring-barbs, tridents, tri-tips, and tri-tips with a ground, with each probe 120 sharpened so as to penetrate or make contact with the patient's skin as described herein. In some embodiments the probe 120 tips penetrate the epidermis and extend to the interface between the epidermis and the dermis, generally in the vicinity of the papillary and reticular layers. However, other depths of probe 120 penetration are also contemplated.

As described, the probe 120 tip forms a point to at least some degree, with an optional insulating layer covering the electrically conductive material of the probe 120 to the position where the probe 120 tip starts to narrow. In some embodiments the probe 120 tip is quite sharp and can penetrate the epidermis of the patient. In other embodiments, the probe 120 tip is not sharp enough to penetrate the epidermis, but the point formed in the probe 120 tip is beneficial for keeping the probe 120 tip in a desired location in the epidermis of the patient.

In some embodiments a plug-in harness 306 connects insulated leads 204 to the device 100, which are connected to the probes 120. In some embodiments, leads 204 to the probes 120 are individually connected to the device 100, but are bundled together with a cable jacket, so as to make lead management easier. In some embodiments, the jacket encasing all of the leads includes shielding for protecting the lead wires 204 and the signals that they carry from external radiation, which might aberrate the signals on the leads 204. Further, in some embodiments, the leads 204 themselves are additionally shielded, so as to not receive interference from the other signal leads 204 nearby or bundled within a common sheath. In some embodiments, magnetic connections between the device 100 and the leads 204 that connect to the probes 120 are provided, for ease in making the electrical connections. In some embodiments, the leads 204 are color-coded for easier placement by the health care provider, such as a black ground lead 204, and red, white, and blue signal leads 204.

A random-access memory module 112 provides short-term storage for data, such as programming instructions for the operation of the device 100, and input data from the sensors 116. A power module 114 is also provided in various embodiments of the device 100. In some embodiments that power module 114 includes at least one of a replaceable battery, a plug-in rechargeable battery, a coil for receiving a wireless charge, and a wirelessly-rechargeable battery that can harvest energy from various signals, such as wi-fi or other electromagnetic signals that might be present in the environment of the device 100.

In some embodiments the steps of the various functions described herein are embodied in a computer language on a non-transitory, computer-readable, data storage medium 106 that is readable by the device 100 of FIG. 1, and that enables the device 100 to implement the functions as described herein, such as a memory card or chip.

### Specific Embodiments

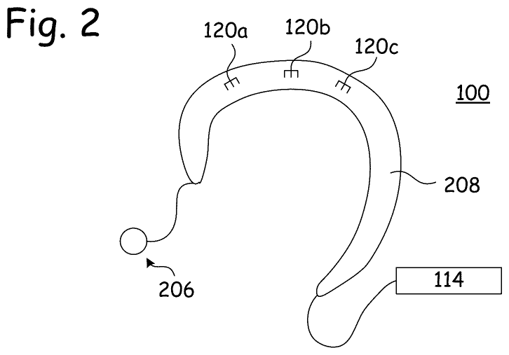

With reference now to FIG. 2 there is depicted a device 100 according to an embodiment of the present disclosure. In this embodiment the device 100 takes the form of an apparatus that can be worn around and behind the patient's ear. In some embodiments the device 100 is adjustable, such as by at least one of length and curvature, so as to more reliably reside behind the ear, or for the comfort of the patient. Various probes 120a-c are disposed along the length of the earpiece 208, for delivery of the signals as described elsewhere herein. A reference or ground contact 206 is also affixed in some manner to the epidermis of the patient, such as by a conductive glue or other means. A power source 114, such as a battery, can power the earpiece 208, either locally such as from a collar location, or secured behind the patient's ear, or in a more distant location such as a pocket.

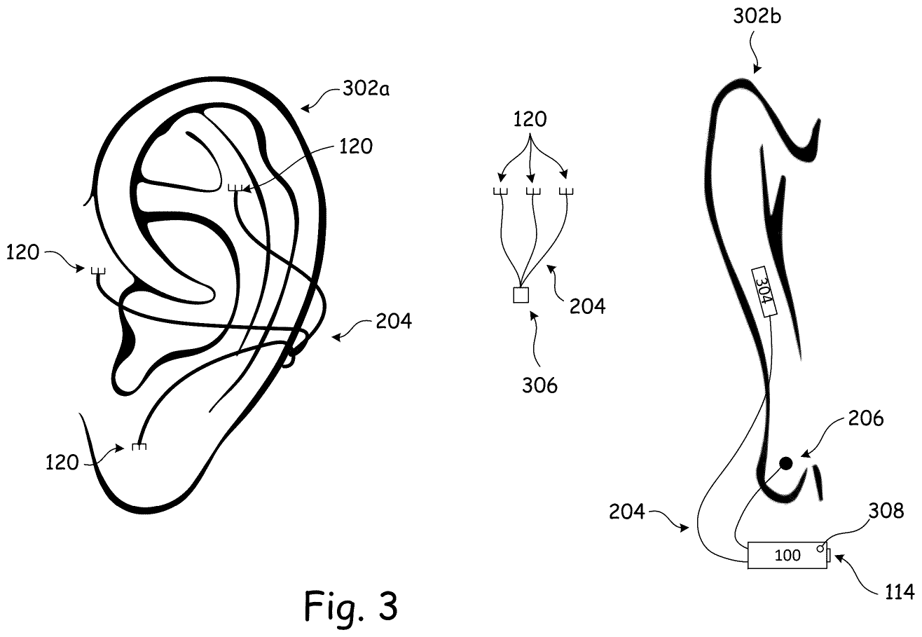

With reference now to FIG. 3 there is depicted a placement diagram for a device 100 according to an embodiment of the present disclosure. This embodiment shows possible probe 120 placement locations, wiring harness 304 location, and device 100 placement location on the front 302a and rear 302b of a patient's ear. Also depicted is the power indicator 308, as described elsewhere herein, and power supply 114 source or connection point. In this depiction, one portion of a wiring harness 306 attaches to and forms an electrical connection with the other portion of the wiring harness 304. The attachment is, in various embodiments, at least one of a magnetic attachment or a latched attachment.

### Artificial Intelligence

In some embodiments, the device 100 reads the patient's autonomic nervous system via at least one of HRV and other physiological data as described elsewhere herein. The data is analyzed by AI, which sets the stimulation parameters as applied to one or more of the probes 120, such as at least one of location of signal delivery, signal intensity, signal waveform shape, signal cadence, signal duration, and signal amplitude. The purpose of the signal as delivered on one or more of the probes 120 is to stimulate the nervous system so as to interrupt, attenuate, or otherwise block any pain that the patient is feeling, as detected by the various inputs.

In some embodiments, the AI that is used is a more traditional rules-based system, in which a health care provider determines the stimulation parameters that should be used as input, and writes those parameters into a table where given inputs always lead to predetermined outputs on the probes 120. In other embodiments a more autonomous AI system is employed, where the AI logic interrogates and learns from a training database of relevant heart rate variability and other possible input parameters, and is able to arrive at an output set of stimulation parameters based at least in part upon what the AI views as the more relevant data from the training database, which output set of stimulation parameters may or might not exactly match anything that has been previously tried.

These embodiments tend to require a greater amount of memory for both computation and data, and more capable processing hardware. Some type of hardware cooling can also be beneficial in some embodiments.

The AI programming can be triggered and changed by specific data sent to the device by commonly used devices such as a smart watch, a wearable ECG device, or specific data gathering devices, that monitor heart rate, HRV, ECG, and other parameters, such as blood pressure, blood oxygen content, and skin resistivity. The AI controls the vagal nerve stimulation to control and balance the parasympathetic nervous system (PNS) and the sympathetic nervous system (SNS) of the autonomic nervous system (ANS). The wearable data source device measures the heart rate and ECG and transmits this data to the nerve stimulating device. The device 100 converts this data to HRV every few minutes, in some embodiments. The device 100, with its AI software, adjusts the stimulation parameters (as discussed elsewhere herein) to optimize the HRV, and thus use that as a feedback for lowering the pain sensation of the patient.

In some embodiments, the signal probes 120 are connected to positions on and around the patient's ear, so as to stimulate the auriculotemporal nerve, the trigeminal nerve, the vagal nerve,

In various embodiments, HRV is measured by at least one of a photoplethysmography method, and an electrocardiogram method. In some embodiments, the data source device, in the embodiments where it is external to the device 100, transmits data, such as heart rate and other data, wirelessly to the device 100.

### Additional Descriptions

Without being bound by any theories of operation as may be postulated and described in this section, various additional embodiments are described and contemplated by the information provided hereinafter.

The device 100 is effective for treating the acute pain occurring from opioid withdrawal, although it is not a cure for addiction.

The electrical stimulation may enhance the release of neuropeptides, primarily beta-endorphin and enkephalins in the central nervous system. These neuropeptides then bind to the vacant mu and delta receptors thereby alleviating the symptoms of physical withdrawal. The device 100 is used to detoxify an opioid-dependent patient without the patient experiencing the discomfort of withdrawal symptoms. Patients are then subjected to cognitive behavioral therapy and extended-release naltrexone injections or implants after detoxification.

The successful treatment of opioid-dependent patients is challenging and requires addressing multiple issues. For those patients that are motivated to be opioid-free, their most significant initial obstacle is the anxiety of the intense physical discomfort of detoxification.

Patients who are good candidates include those who are using only because they are afraid of withdrawal sickness. They are no longer using for the explicit purpose of getting high. When these patients see a video of a detoxing patient actually change, within minutes, from the uncomfortable appearance of detoxification to smiling and comfortable, they are willing to proceed with the important first step in the process on the road to becoming opioid-free.

This device is an effective tool to detoxify opioid-dependent patients comfortably by causing an increased availability of endogenous neuropeptides that occupy vacant opioid receptors.

The device 100 allows for adjustable power settings including an increase of power to the auricular nerve stimulation. The device 100 can be functionally tested at any time, including while still on the patient's ear, to ensure that it is operating properly. The device 100 stimulates the nerves percutaneously to aid in the reduction of withdrawal symptoms associated with substance use disorder.

The electrical stimulation enhances the release of neuropeptides, primarily beta-endorphin and enkephalins in the central nervous system. These neuropeptides then bind to the vacant mu and delta receptors thereby alleviating the symptoms of physical withdrawal. The device is used to detoxify opioid dependent without the patient experiencing the discomfort of withdrawal symptoms.

The device 100 supplies an electrical signal to the nervous system of a patient. This signal can be a DC signal (or voltage), either positive or negative, a square wave signal (or voltage), which is positive and negative or a sinusoidal signal (or voltage), which is positive or negative. It can vary in amplitude, frequency, and duration. The purpose of this electrical injection or signal into the nervous system of a patient, is to use the nerves as an electrical conductor that will cause a reaction in the body. This reaction can be to modify the electrical signals sent from the brain, to alter these signals, to block these signals, or to cause a specific chemical reaction to occur in the body or to not occur in the body.

The device has electronics, batteries, a system to communicate with other electronic devices, a system to program the device, a system to communicate when the batteries are low (Such as an LED), and an electrical system (wires, probes, connectors, insulation) that will deliver the desired voltage (signal) to specific nerves in the body. The device 100 delivers and monitors the voltage, as required by a health care provider, to achieve the desired results in the body. The device 100 can be a single device or two or more devices communicating with each other. One device can be located on the ear, with a separate device connected by wires that contains the battery and the electronics.

The portion of the device 100 on the ear can have single point probes or multiple point probes. These probes can be movable. This mobility would allow them to be moved to allow the health care provider to find the specific neurovascular bundles she is looking for. Any combination of devices and functionality are envisioned in this disclosure.

The batteries can function for a specific period of time. In one embodiment the batteries last for four or five days. In another embodiment the batteries power the device 100 for fifteen days. The battery capacity can be specific to the condition of the patient that the health care provider is trying to treat. Having different battery capacity can help to mitigate the cost of the device 100. The entire system of probes, wiring connectors, electronics, charging, and communication ports can be water resistant, allowing for the patient to clean themself.

One embodiment of the programming sequence is: the device 100 plugs into an interface, a voltage reading appears, and the interface is connected to a computing device, such as an iPad. Programming of the device 100 occurs via the iPad. In another embodiment: an iPAD or smart phone, with a specific application replaces the interface. In another embodiment: a wi-fi adaptor is plugged into a port on the device temporarily for programming and troubleshooting (HIPPA compliant). The display on the iPad or smart phone provides an oscilloscope function for waveform, frequency, pulse width, and amplitude for verification after programming is completed.

The adhesive that attaches the device 100 to the skin is a medically approved adhesive, such as a medical adhesive.

The programming device can use the default setting touch screen parameters for current FDA approved settings, but also have the ability for a practitioner to customize days of stimulation, intensity, and duty cycle. The system can keep ear map stimulation points for various disease treatments as well as keeping a list of diseases and stimulation recommendations. Programming can allow for each electrode to be programmed with the same electrical stimulus, or each electrode to be programmed with a different electrical stimulus.

The device 100 is unique because it delivers a specific electrical signal or stimulation to multiple nerves simultaneously or sequentially or in any order, with the specific voltage shape (amplitude, frequency, duration, shape) to create the desired result within the human body. This is accomplished by first diagnosing the problem within the patient and then developing and deploying the proper electrical protocol to modify or eliminate the condition of the patient.

The device 100 allows the health care provider to deploy all variations of electrical stimulus to any nerves in the body. One embodiment can be to locate and stimulate the neurovascular bundles in and around the ear. Locating the neurovascular bundles can be by a transillumination method. However, an impedance, resistance, or voltage drop method could also be used for locating neurovascular bundles.

The electronics can allow for multiple wires to be connected at the same time, with each wire having the ability to deliver a different or the same signal to the probes that attach to the skin of the patient at the sight of the specific nerve endings. The wires can be insulated and color coded, and have one pointed probe or multiple pointed probes at the end of the wire to make contact with the skin, such as at the point that is most readily accessible to the specific nerve required.

The probes can also be insulated, up to the tip, and color coded as well. The probes and wiring can be soldered to the electronic circuit board, or be a separate item that is connected to the electronics via an electrical connector. The tips, wiring, and connector can be sterilized. This can be accomplished with heat and pressure, such as in an autoclave, or through the use of UVC lighting, or through the use of ozone or other sterilizing liquids such as alcohol.

An example of a multiple probe is a trident probe having three points with a certain distance separating the pointed tips. This enables the resistance of the skin to be reduced, thus supplying more current to the specific nerve in the body. Depending upon the current required, a lower voltage could be required, thus extending the battery life of the device 100. The resistance calculation of the trident tip to the end of the nerve can be calculated as follows: Rtotal=1/(1/R1+1/R2+1/R3).

In one embodiment, the skin has a resistance of 10 ohms. A voltage of 5 volts is provided. A single probe and its electronics then deliver 0.5 amps to the nerve (V=IR, voltage equals current times resistance, in order to solve for current (I), the equation becomes I=V/R, or current equals voltage divided by resistance). However, if three probes contact the skin, the resistance becomes Rtotal=1/( 1/10+ 1/10+ 1/10) or a resentence of 3.33 ohms. This decrease in resistance allows a lower voltage to supply the same current to the nerve. Voltage-Current times Resistance (V=IR, 1.66 volts=0.5×3.33). With current flow in the nerve being the important factor, the trident lowers the skin resistance and, therefore, delivers the same current with less power. Thus, the batteries last longer. The trident tip also makes it much easier to locate the desired nerve ending (three chances to find the nerve ending). The battery, with the voltage and the power it contains, is important because it extends the life of the device 100 before the batteries must be replaced or recharged. This is important to the patient. Another advantage of this device 100 is to have the wires made of copper to decrease the resistance in the wires and to deliver more power (Power=Voltage times Current) to the nervous system in the body.

The device 100 can be used to treat pain from, but is not limited to pain from, post op shoulder surgery, post op knee surgery, post op cardiovascular surgery, post op caesarian section surgery, drug detox, migraine headaches, pediatric irritable bowel, and diabetic neuropathy.

### Endogenous Opioid Circuits

| Endorphin | Receptor | Frequency (Hz) | Location |
|---|---|---|---|
| NK cells | Immune | 4 | Widespread |
| Beta-endorphins | μ | 2-4 | Mid-brain/PAG |
| Enkephalins | δ | 2-4/15 | Dorsal Horn |
| Dynorphins | κ | 100 | Brainstem/Spine |
| Orphanin | μ | 2/15 | Widespread CNS |
| 5-HTP | 5-HTPF | 20-50 | Hypothalamus |
| Oxytocin | OXTR | 2-15/30 | CNS |
| Dopamine | D1 | 2, 15-30 | Prefrontal |
| NOS | Epithelium | 2, 15-30 | Widespread |

Natural killer cells, also known as NK cells or large granular lymphocytes (LGL), are a type of cytotoxic lymphocyte critical to the innate immune system that belong to the rapidly expanding family of known innate lymphoid cells (ILC) and represent 5-20% of all circulating lymphocytes in humans. The role of NK cells is analogous to that of cytotoxic T cells in the vertebrate adaptive immune response. NK cells provide rapid responses to virus-infected cell and other intracellular pathogens acting at around three days after infection, and respond to tumor formation.

Typically, immune cells detect the major histocompatibility complex (MHC) presented on infected cell surfaces, triggering cytokine release, causing the death of the infected cell by lysis or apoptosis. NK cells are unique, however, as they have the ability to recognize and kill stressed cells in the absence of antibodies and MHC, allowing for a much faster immune reaction. They were named “natural killers” because of the notion that they do not require activation to kill cells that are missing “self” markers of MHC class 1. This role is especially important because harmful cells that are missing MHC I markers cannot be detected and destroyed by other immune cells, such as T lymphocyte cells.

In addition to natural killer cells being effectors of innate immunity, both activating and inhibitory NK cell receptors play important functional roles, including self-tolerance and the sustaining of NK cell activity. NK cells also play a role in the adaptive immune response: numerous experiments have demonstrated their ability to readily adjust to the immediate environment and formulate antigen-specific immunological memory, fundamental for responding to secondary infections with the same antigen. The role of NK cells in both the innate and adaptive immune responses is becoming increasingly important in research using NK cell activity as a potential cancer therapy.

There are three well-characterized families of opioid peptides produced by the body: enkephalins, B-endorphin, and dynorphins.

β-Endorphin is an agonist of the opioid receptors; it preferentially binds to the μ-opioid receptor. It serves as a primary endogenous ligand for the μ-opioid receptor, the same receptor to which the chemicals extracted from opium, such as morphine, derive their analgesic properties. β-Endorphin function is said to be divided into two main categories: local function and global function. Global function of β-endorphin is related to decreasing bodily stress and maintaining homeostasis resulting in pain management, reward effects, and behavioral stability. Localized function of β-endorphin results in release of β-endorphin in different brain regions such as the amygdala or the hypothalamus.

An enkephalin is a pentapeptide involved in regulating nociception (pain sensation) in the body.

Dynorphin is a neuropeptide involved in pain, addiction, and mood regulation. It exerts its activity by binding to the kappa opioid receptor (KOP) which belongs to the large family of G protein-coupled receptors.

Orphanin—The NOP receptor (nociceptin/orphanin FQ opioid peptide receptor) is the most recently discovered member of the opioid receptor family and, together with its endogenous ligand, N/OFQ, make up the fourth members of the opioid receptor and opioid peptide family. NOP receptor activation has a clear modulatory role on mu opioid receptor-mediated actions and thereby affects opioid analgesia, tolerance development, and reward. In addition to opioid modulatory actions, NOP receptor activation has important effects on motor function and other physiologic processes.

5-HT receptors, 5-hydroxytryptamine receptors, or serotonin receptors, are a group of G protein-coupled receptor and ligand-gated ion channels found in the central and peripheral nervous systems. They mediate both excitatory and inhibitory neurotransmission. The serotonin receptors are activated by the neurotransmitter serotonin, which acts as their natural ligand. The serotonin receptors modulate the release of many neurotransmitters, including glutamate, GABA, dopamine, epinephrine/norepinephrine, and acetylcholine, as well as many hormones, including oxytocin, prolactin, vasopressin, cortisol, corticotropin, and substance P, among others. Serotonin receptors influence various biological and neurological processes such as aggression, anxiety, appetite, cognition, learning, memory, mood, nausea, sleep, and thermoregulation. They are the target of a variety of pharmaceutical and recreational drugs, including many antidepressants, antipsychotics, anorectics, antiemetics, gastroprokinetic agents, antimigraine agents, hallucinogens, and entactogens.

Oxytocin is a hormone that's produced in the hypothalamus and released into the bloodstream by the pituitary gland. Its main function is to facilitate childbirth, which is one of the reasons it is called the “love drug” or “love hormone.”

Dopamine is a type of neurotransmitter. Your body makes it, and your nervous system uses it to send messages between nerve cells. That's why it's sometimes called a chemical messenger. Dopamine plays a role in how we feel pleasure. It's a big part of our unique human ability to think and plan. It helps us strive, focus, and find things interesting.

NOS—Nitric oxide synthases (NOSs) are a family of enzymes catalyzing the production of nitric oxide (NO) from L-arginine. NOS activity has also been correlated with major depressive episodes (MDEs) in the context of major depressive disorder. Patients in a major depressive episode have significantly lower NOS activity compared to healthy patients, whilst treatment with antidepressants significantly elevated NOS activity levels in patients in a major depressive episode.

In one embodiment, the device 100 is used as given below:
1) The stimulation sites on the auricle (ear) are selected and marked with an ink marker.
2) The sterile trident pins are removed from packaging and applied to the marked areas for stimulation.
3) The electrode harness array is plugged into the device and activated and programmed with the desired stimulation parameters.
4) The distal end of each electrode is a pan-head magnetic electrically-conductive metal.
5) The pan-head magnetic end of each color-coded electrode connects to the appropriate pin to complete the connections.
6) The fourth electrode serves as the ground and connects in the same fashion to a pin placed behind the ear lobe.
7) A small circular adhesive-type bandage is applied to each electrode connection for additional security.

The electrode wires are formed of malleable copper, but resistant to breaking. The wires can be bent to customize their position on the patient (around curves and tucked away).

Verification of the electrical integrity all the way to the pins and an oscilloscope verification of the programmed settings is provided in some embodiments.

When the device 100 is applied to the patient, the health care provider reads the patient's autonomic nervous system via heart rate variability similar to the ansiscope. The data is analyzed by AI and it sets the stimulation parameters.

As used herein, the phrase “at least one of A, B, and C” means all possible combinations of none or multiple instances of each of A, B, and C, but at least one A, or one B, or one C. For example, and without limitation: A×1, A×2+B×1, C×2, A×1+B×1+C×1, A×7+B×12+C×113. It does not mean A×0+B×0+C×0.

The foregoing description of embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A nerve stimulation device, comprising:
a processor adapted to receive input parameters pertaining to physiological conditions of a patient, and to processes the input parameters using AI to specify output parameters of a signal,
a waveform generator adapted to receive the output parameters and to create the signal having the output parameters,
an amplifier adapted to receive the signal and to amplify it to a desired level,
an output adapted to receive the signal from the amplifier,
a lead adapted to receive the signal from the output, and
a probe adapted to receive the signal from the lead and to deliver the signal to a nerve of the patient.

2. The nerve stimulation device of claim 1, wherein the physiological conditions include at least one of temperature, heart rate, HRV, blood pressure, electrical impulses of the patient's nervous system, blood oxygenation, hydration, and brain activity.

3. The nerve stimulation device of claim 1, further comprising sensors for measuring the physiological conditions.

4. The nerve stimulation device of claim 1, further comprising a radio for receiving at least one of the physiological conditions, power for the device, and operating instructions.

5. The nerve stimulation device of claim 1, wherein the lead comprises a signal lead and a ground lead.

6. The nerve stimulation device of claim 1, wherein the lead comprises multiple leads and the signal is applied to each of the multiple leads.

7. The nerve stimulation device of claim 1, wherein the lead comprises multiple leads and a different signal is applied to each of the multiple leads.

8. The nerve stimulation device of claim 1, wherein the output parameters include at least one of location of signal delivery, signal intensity, signal waveform shape, signal frequency, signal cadence, signal duration, and signal amplitude.

9. The nerve stimulation device of claim 1, wherein the probe is a tri-tip probe that pierces the patient's epidermis and delivers the signal at an interface between the patient's epidermis and dermis.

10. The nerve stimulation device of claim 1, wherein the probe delivers the signal to at least one of the patient's auriculotemporal nerve, trigeminal nerve, and vagal nerve.

11. A method of reducing pain in a patient, the method comprising the steps of:
receiving input parameters pertaining to physiological conditions of the patient, and processing the input parameters using AI to specify output parameters of a signal,
receiving the output parameters with a waveform generator and creating the signal having the output parameters,
receiving the signal with an amplifier and amplifying the signal to a desired level,
receiving the signal from the amplifier with an output,
receiving the signal from the output with a lead,
receiving the signal from the lead with a probe, and
delivering the signal from the probe to a nerve of the patient, the signal providing neuromodulation to the nerve and reducing the pain in the patient.

12. The method of claim 11, wherein the physiological conditions include at least one of temperature, heart rate, HRV, blood pressure, electrical impulses of the patient's nervous system, blood oxygenation, hydration, and brain activity.

13. The method of claim 11, further comprising measuring the physiological conditions with sensors.

14. The method of claim 11, further comprising receiving at least one of the physiological conditions, power for the device, and operating instructions with a radio.

15. The method of claim 11, the signal is applied to each of multiple leads.

16. The method of claim 11, wherein a different signal is applied to each of multiple leads.

17. The method of claim 11, wherein the output parameters include at least one of location of signal delivery, signal intensity, signal waveform shape, signal frequency, signal cadence, signal duration, and signal amplitude.

18. The method of claim 11, the signal is delivered at an interface between the patient's epidermis and dermis.

19. The method of claim 11, wherein the signal is delivered to at least one of the patient's auriculotemporal nerve, trigeminal nerve, and vagal nerve.

20. The method of claim 11, wherein the pain includes pain from at least one of post operative shoulder surgery, post operative knee surgery, post operative cardiovascular surgery, post operative caesarian section surgery, drug detox, migraine headaches, pediatric irritable bowel, and diabetic neuropathy.
